(19) United States
(12) Patent Application Publication
(10) Pub. No.: US 2025/0176587 A1
(43) Pub. Date: Jun. 5, 2025

(54) PROBIOTIC-CONTAINING SEA CUCUMBER PEPTIDE POWDER FOR IMPROVING IMMUNITY AND PREPARATION METHOD THEREOF

(71) Applicants: TIANJIN UNIVERSITY OF SCIENCE AND TECHNOLOGY, Tianjin (CN); TIANJIN JIANLI TECHNOLOGY R & D CO., LTD., Tianjin (CN)

(72) Inventors: Tao WU, Tianjin (CN); Gengxiao HAN, Tianjin (CN); Yongde CHEN, Tianjin (CN); Xiaoman CI, Tianjin (CN); Guoxing CHEN, Tianjin (CN); Min ZHANG, Tianjin (CN)

(73) Assignees: TIANJIN UNIVERSITY OF SCIENCE AND TECHNOLOGY, Tianjin (CN); TIANJIN JIANLI TECHNOLOGY R & D CO., LTD., Tianjin (CN)

(21) Appl. No.: 18/648,449

(22) Filed: Apr. 28, 2024

(30) Foreign Application Priority Data
Dec. 5, 2023 (CN) 202311659215.X

(51) Int. Cl. A23J 1/04; A23L 33/135

(52) U.S. Cl. CPC A23J 1/04; A23L 33/135

(57) ABSTRACT

The present disclosure provides a probiotic-containing sea cucumber peptide powder for improving an immunity and a preparation method thereof and belongs to the technical field of food processing. In the present disclosure, papain and a neutral protease are used in combination to allow step-by-step enzymatic hydrolysis, and Eurotium cristatum and/or Bacillus coagulans are/is used to allow fermentation, so as to prepare a probiotic-containing sea cucumber peptide powder without a fishy smell. Enzyme inactivation and decolorization are not required throughout the preparation method of the present disclosure. The probiotic-containing sea cucumber peptide powder prepared by the preparation method does not have a fishy smell, is light-yellow with a bright luster, and has a high live probiotic content. The probiotic-containing sea cucumber peptide powder is suitable for daily health care for people with a low immunity.

### CROSS-REFERENCE TO RELATED APPLICATIONS

This patent application claims priority to Chinese Patent Application No. 202311659215.X, filed with the China National Intellectual Property Administration on Dec. 5, 2023, the disclosure of which is incorporated by reference herein in its entirety as part of the present application.

### BIOLOGICAL DEPOSIT

A Bacillus coagulans strain, which has an Accession number: CGMCC No. 21801, wherein said Accession number was obtained by depositing said strain on Feb. 1, 2021, in the China General Microbiological Culture Collection Center, the Institute of Microbiology, Chinese Academy of Sciences, at the address: No. 3, Courtyard 1, West Beichen Road, Chaoyang District, Beijing City. The details of the Bacillus coagulans strain are set forth below.

### TECHNICAL FIELD

The present disclosure relates to the field of aquatic product processing, and specifically relates to a probiotic-containing sea cucumber peptide powder for improving an immunity and a preparation method thereof.

### BACKGROUND

Sea cucumbers have been used as a superior nutritional health food since ancient times. Sea cucumbers have the effects of enhancing a muscle strength, enhancing an immunity, fighting against a tumor, preventing arthritis, and lowering a blood lipid level. Proteins including many types of collagen are an important part of a sea cucumber, and a protein content in a dried sea cucumber is close to 90%. Sea cucumber peptides refer to small-molecule peptides produced through protease hydrolysis of a sea cucumber, separation, and purification. A sea cucumber includes a variety of active peptides such as neuropeptides, glycopeptides, and antimicrobial peptides. Sea cucumber peptides have a variety of effects and can play an irreplaceable role in physiological functions of a human body. Because sea cucumber peptides have special physical and chemical properties such as excellent solubility, high stability, low viscosity, easy digestion and absorption, no antigenicity, and food safety, sea cucumber peptides have a higher biological potency than other ordinary sea cucumber products.

A fishy smell of sea cucumber peptides is an important factor affecting an acceptance of a sea cucumber peptide product, and methods for removing the fishy smell mainly include physical methods such as adsorption, masking, and embedding. However, the adsorption method will cause the loss of an original flavor of sea cucumbers. The masking and embedding methods will make a proportion of sea cucumber components relatively small, and sea cucumber peptide products produced by the masking and embedding methods will become fishy once again after being stored for a period of time.

Body walls of sea cucumbers are mostly brown or dark-brown, and include a large number of pigments. Thus, a protease hydrolysate of a sea cucumber is dark-gray, which affects the appearance of a product, seriously affects a quality of a product, and hinders the in-depth development of a product. Therefore, it is important to decolorize a sea cucumber polypeptide-containing enzymatic hydrolysate, which is conducive to the in-depth development of a product. Currently, decolorizing agents such as powdered activated carbon, granular activated carbon, diatomaceous earth, macroporous absorption resins, composite adsorbents, weakly-alkaline anionic resins, strongly-alkaline anionic resins, and weakly-acidic cationic resins are often used to decolorize sea cucumber polypeptides. However, the traditional decolorization process is complicated, has a high loss rate, and will destroy original structures and activities of sea cucumber peptides.

### SUMMARY

The present disclosure provides a preparation method of a probiotic-containing sea cucumber peptide powder for improving an immunity. The probiotic-containing sea cucumber peptide powder prepared by the preparation method does not have a fishy smell, is light-yellow with a bright luster, has a high live probiotic content, and retains an activity of a sea cucumber peptide powder to the maximum extent. In addition, enzyme inactivation and decolorization are not required throughout the preparation method, such as addition of an adsorbent, activated carbon, and the like.

The present disclosure provides a preparation method of a probiotic-containing sea cucumber peptide powder for improving an immunity, at least including the following steps:
(1) to a homogenate of a body wall of a sea cucumber, adding a neutral protease to allow first enzymatic hydrolysis, and then adding papain to allow second enzymatic hydrolysis to obtain an enzymatic hydrolysate; and
(2) adding a probiotic to the enzymatic hydrolysate to allow fermentation, and collecting a supernatant produced after the fermentation,
where the probiotic in the step (2) is Eurotium cristatum with an accession number of CICC No. 2099 and/or Bacillus coagulans with an accession number of CGMCC No. 21801.

Further, the homogenate of the body wall of the sea cucumber refers to a slurry obtained through cutting the body wall of the sea cucumber into pieces, adding water, and homogenizing. In the present disclosure, the homogenate of the body wall of the sea cucumber can be prepared as follows: washing a dried sea cucumber, cutting a washed sea cucumber into small pieces, adding water, and beating to obtain a slurry; and homogenizing and filtering the slurry.

Specifically, the neutral protease is added to the homogenate of the body wall of the sea cucumber to allow first enzymatic hydrolysis for 1 h to 2 h at 50° C. to 55° C. and a pH of 6.5 to 7.5; and then the papain is added to allow second enzymatic hydrolysis for 2 h to 3 h at 50° C. to 55° C. and a pH of 6.5 to 7.5, where a total enzymatic hydrolysis time is 3 h to 5 h.

Further, in the step (1), an enzymatic activity ratio of the neutral protease to the papain is (1-5):1.

Preferably, in the step (1), the enzymatic activity ratio of the neutral protease to the papain is 1:1.

Further, in the step (1), a total amount of the neutral protease and the papain is 6,000 U/g to 9,000 U/g, and the first and second enzymatic hydrolysis are conducted for 3 h to 5 h in total at 50° C. to 55° C. and a pH of 6.5 to 7.5.

Specifically, in the step (2), the probiotic is inoculated into a fermentation tank to allow the fermentation, where the probiotic is Eurotium cristatum (accession number: CICC No. 2099), Bacillus coagulans (accession number: CGMCC No. 21801), or a mixture of Eurotium cristatum and Bacillus coagulans.

Further, in the step (2), an amount of the Eurotium cristatum is 0.02% to 0.04% of a mass of the enzymatic hydrolysate.

Further, in the step (2), an amount of the Bacillus coagulans is 0.02% to 0.04% of a mass of the enzymatic hydrolysate.

Further, in the step (2), the fermentation is conducted at 36° C. to 38° C. for 40 min to 60 min.

Further, in the step (2), the probiotic is a mixture of Eurotium cristatum and Bacillus coagulans, and a colony-forming unit (CFU) ratio of the Eurotium cristatum to the Bacillus coagulans is 1:(1-3); and a total amount of the Eurotium cristatum and the Bacillus coagulans is 0.02% to 0.04% of a mass of the enzymatic hydrolysate.

Further, in the step (2), the probiotic is a mixture of Eurotium cristatum and Bacillus coagulans, and the Eurotium cristatum and the Bacillus coagulans can be added as follows: the Eurotium cristatum and the Bacillus coagulans are first mixed and then added to the enzymatic hydrolysate to allow the fermentation; or the Eurotium cristatum and the Bacillus coagulans are added separately to the enzymatic hydrolysate to allow the fermentation.

Further, the preparation method further includes: concentrating the supernatant collected in the step (2), and spray-drying a concentrate at a low temperature to obtain the high-quality probiotic-containing sea cucumber peptide powder.

The present disclosure also provides a probiotic-containing sea cucumber peptide powder prepared by the preparation method described above.

The technical solutions of the present disclosure have the following advantages:

1. Fishy smell removal effect: After the sea cucumber enzymatic hydrolysate produced after full enzymatic hydrolysis is subjected to synergistic fermentation with Eurotium cristatum and Bacillus coagulans, a relative content of 9 representative fishy substances in the sea cucumber peptide powder is effectively reduced from 49.97% to 2.78%.

2. Decolorization effect: After the synergistic fermentation with Eurotium cristatum and Bacillus coagulans, the sea cucumber enzymatic hydrolysate turns from the original brown solution into a light-yellow transparent liquid, and a supernatant produced after the synergistic fermentation is collected, concentrated, and spray-dried to obtain a milky-yellow sea cucumber peptide powder with a bright luster. The decolorization effect is shown in FIGS. 2A-2D. This technical solution omits a decolorization process, which allows simple operations and reduces a cost.

3. Peptide powder activity: After the homogenate of the body wall of the sea cucumber is subjected to enzymatic hydrolysis successively with the neutral protease and the papain, high-temperature enzyme inactivation is not required, but Eurotium cristatum and Bacillus coagulans are directly used to allow synergistic fermentation with the proteases as nutrients. After the synergistic fermentation, there is no need for sterilization, and a resulting fermentation broth is directly spray-dried at a low temperature, which retains live probiotics and allow a live probiotic content of greater than 1×10⁹/g. Moreover, the whole process is completed at a low temperature, which retains an activity of the sea cucumber peptide powder to a maximum extent and can significantly improve an immunity of a body.

4. Flavor sense: After the sea cucumber enzymatic hydrolysate is subjected to synergistic fermentation with Eurotium cristatum and Bacillus coagulans, a fishy smell is well removed, and pleasant fermentation and mellow aromas are produced, resulting in high sensory evaluation.

### BRIEF DESCRIPTION OF THE DRAWINGS

FIG. 1 shows impacts of fermentation of different strains on a sensory score and a fishy smell level of a sea cucumber peptide-containing enzymatic hydrolysate, where a shows a sensory score and a fishy smell level of a sea cucumber peptide-containing enzymatic hydrolysate fermented by Lactobacillus acidophilus; b shows a sensory score and a fishy smell level of a sea cucumber peptide-containing enzymatic hydrolysate fermented by Eurotium cristatum; c shows a sensory score and a fishy smell level of a sea cucumber peptide-containing enzymatic hydrolysate fermented by Bifidobacterium longum; d shows a sensory score and a fishy smell level of a sea cucumber peptide-containing enzymatic hydrolysate fermented by Lactobacillus casei; e shows a sensory score and a fishy smell level of a sea cucumber peptide-containing enzymatic hydrolysate fermented by Lactobacillus gasseri; f shows a sensory score and a fishy smell level of a sea cucumber peptide-containing enzymatic hydrolysate fermented by Bacillus coagulans; and g shows a sensory score and a fishy smell level of a sea cucumber peptide-containing enzymatic hydrolysate co-fermented by Eurotium cristatum and Bacillus coagulans;

FIGS. 2A, 2B, 2C and 2D show pictures of sea cucumber peptide powders with a fishy smell removed, where FIG. 2A is a picture of a sea cucumber peptide powder with a fishy smell removed by synergetic fermentation of Eurotium cristatum and Bacillus coagulans; FIG. 2B is a picture of a sea cucumber peptide powder with a fishy smell removed by fermentation of Eurotium cristatum; FIG. 2C is a picture of a sea cucumber peptide powder with a fishy smell removed by fermentation of Bacillus coagulans; and FIG. 2D is a picture of an unfermented sea cucumber peptide powder;

FIG. 3 shows a high-performance liquid chromatography spectrum of a sea cucumber peptide powder without a fishy smell in an embodiment of the present disclosure;

FIG. 4 shows protein recovery rates and hydrolysis degrees of sea cucumber peptides produced after hydrolysis with a neutral protease added first and then papain added;

FIG. 5 shows protein recovery rates and hydrolysis degrees of sea cucumber peptides produced after hydrolysis with papain added first and then a neutral protease added; and

FIG. 6 shows protein recovery rates and hydrolysis degrees of sea cucumber peptides produced after hydrolysis with a neutral protease and papain added simultaneously.

FIGS. 7A, 7B, 7C, 7D, 7E, and 7F shows Table 1 which provides comparative analysis results of relative percentage contents of volatile components in the four products.

### DETAILED DESCRIPTION

The present disclosure can be further well understood through the following examples, but is not limited to the preferred implementations. The examples do not limit the content and protection scope of the present disclosure. Any product that is the same as or similar to the product of the present disclosure and is obtained in light of the present disclosure or by combining the present disclosure with other features of the prior art shall fall within the protection scope of the present disclosure.

### Example 1 Fermentation with a Mixture of Eurotium cristatum and Bacillus coagulans to Prepare a Sea Cucumber Peptide Powder

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate, which was a homogenate of the body wall of the sea cucumber for later use.

(3) Enzymatic hydrolysis: The filtrate obtained in the step (2) was subjected to step-by-step enzymatic hydrolysis with an enzyme combination as follows: A neutral protease was first added to allow first enzymatic hydrolysis for 1 h at 50° C. and a pH of 7; and then papain was added to allow second enzymatic hydrolysis for 2.5 h at 50° C. and a pH of 7. A total amount of the neutral protease and the papain was 8,000 U/g, and an enzymatic activity ratio of the papain to the neutral protease was 1:1.

(4) Microbial fermentation: An enzymatic hydrolysate obtained in the step (3) was transferred to a fermentation tank, and the direct-vat-set fermentation strains of Eurotium cristatum and Bacillus coagulans were added to allow fermentation at 37° C. for 50 min. A CFU ratio of the Eurotium cristatum to the Bacillus coagulans was 1:1.5, and a total amount of the Eurotium cristatum and the Bacillus coagulans was 0.03% of a mass of the enzymatic hydrolysate.

(5) A fermentation broth obtained after the step (4) was concentrated and then spray-dried at a low temperature to obtain a finished sea cucumber peptide powder product that was rich in Eurotium cristatum and Bacillus coagulans and did not have a fishy smell.

### Example 2 Fermentation with Eurotium cristatum to Prepare a Sea Cucumber Peptide Powder

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate, which was a homogenate of the body wall of the sea cucumber for later use.

(3) Enzymatic hydrolysis: The filtrate obtained in the step (2) was subjected to step-by-step enzymatic hydrolysis with an enzyme combination as follows: A neutral protease was first added to allow first enzymatic hydrolysis for 1 h at 50° C. and a pH of 7; and then papain was added to allow second enzymatic hydrolysis for 1.5 h at 50° C. and a pH of 7. A total amount of the neutral protease and the papain was 8,000 U/g, and an enzymatic activity ratio of the papain to the neutral protease was 1:1.

(4) Microbial fermentation: An enzymatic hydrolysate obtained in the step (3) was transferred to a fermentation tank, and the direct-vat-set fermentation strain of Eurotium cristatum was added to allow fermentation at 37° C. for 50 min. An amount of the Eurotium cristatum was 0.03% of a mass of the enzymatic hydrolysate.

(5) A fermentation broth obtained after the step (4) was concentrated and then spray-dried at a low temperature to obtain a finished sea cucumber peptide powder product that was rich in Eurotium cristatum and did not have a fishy smell.

### Example 3 Fermentation with Bacillus coagulans to Prepare a Sea Cucumber Peptide Powder

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate, which was a homogenate of the body wall of the sea cucumber for later use.

(3) Enzymatic hydrolysis: The filtrate obtained in the step (2) was subjected to step-by-step enzymatic hydrolysis with an enzyme combination as follows: A neutral protease was first added to allow first enzymatic hydrolysis for 1 h at 50° C. and a pH of 7; and then papain was added to allow second enzymatic hydrolysis for 1.5 h at 50° C. and a pH of 7. A total amount of the neutral protease and the papain was 8,000 U/g, and an enzymatic activity ratio of the papain to the neutral protease was 1:1.

(4) Microbial fermentation: An enzymatic hydrolysate obtained in the step (3) was transferred to a fermentation tank, and the direct-vat-set fermentation strain of Bacillus coagulans was added to allow fermentation at 37° C. for 50 min. An amount of the Bacillus coagulans was 0.03% of a mass of the enzymatic hydrolysate.

(5) A fermentation broth obtained after the step (4) was concentrated and then spray-dried at a low temperature to obtain a finished sea cucumber peptide powder product that was rich in Bacillus coagulans and did not have a fishy smell.

### Example 4 Preparation of an Unfermented Sea Cucumber Peptide Powder

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate, which was a homogenate of the body wall of the sea cucumber for later use.

(3) Enzymatic hydrolysis: The filtrate obtained in the step (2) was added to a beaker and subjected to step-by-step enzymatic hydrolysis with an enzyme combination as follows: A neutral protease was first added to allow first enzymatic hydrolysis for 1 h at 50° C. and a pH of 7; and then papain was added to allow second enzymatic hydrolysis for 1.5 h at 50° C. and a pH of 7. A total amount of the neutral protease and the papain was 8,000 U/g, and an enzymatic activity ratio of the papain to the neutral protease was 1:1.

(4) Enzyme inactivation: An enzymatic hydrolysate obtained after the step (3) was subjected to enzyme inactivation as follows: The enzymatic hydrolysate was heated in a boiling water bath at 90° C. for 15 min and then cooled to room temperature.

(5) Centrifugation: An enzyme inactivation system obtained after the step (4) was centrifuged at 3,500 rpm/min for 20 min, and a resulting supernatant was collected.

(6) Concentration: The supernatant collected in the step (5) was concentrated to a volume 5% to 10% of an original volume.

(7) Drying: A concentrate obtained after the step (6) was spray-dried at a low temperature to obtain a finished sea cucumber peptide powder product.

### Example 5 Fermentation with Lactobacillus acidophilus to Prepare a Sea Cucumber Peptide Powder

This example was the same as Example 2, except that the probiotic was Lactobacillus acidophilus.

### Example 6 Fermentation with Bifidobacterium longum to Prepare a Sea Cucumber Peptide Powder

This example was the same as Example 2, except that the probiotic was Bifidobacterium longum.

### Example 7 Fermentation with Lactobacillus casei to Prepare a Sea Cucumber Peptide Powder

This example was the same as Example 2, except that the probiotic was Lactobacillus casei.

### Example 8 Fermentation with Lactobacillus Gasseri to Prepare a Sea Cucumber Peptide Powder

This example was the same as Example 2, except that the probiotic was Lactobacillus gasseri.

### Example 9 Detection and Analysis of Fishy Substances of Sea Cucumber Peptide Powders Prepared by Different Methods

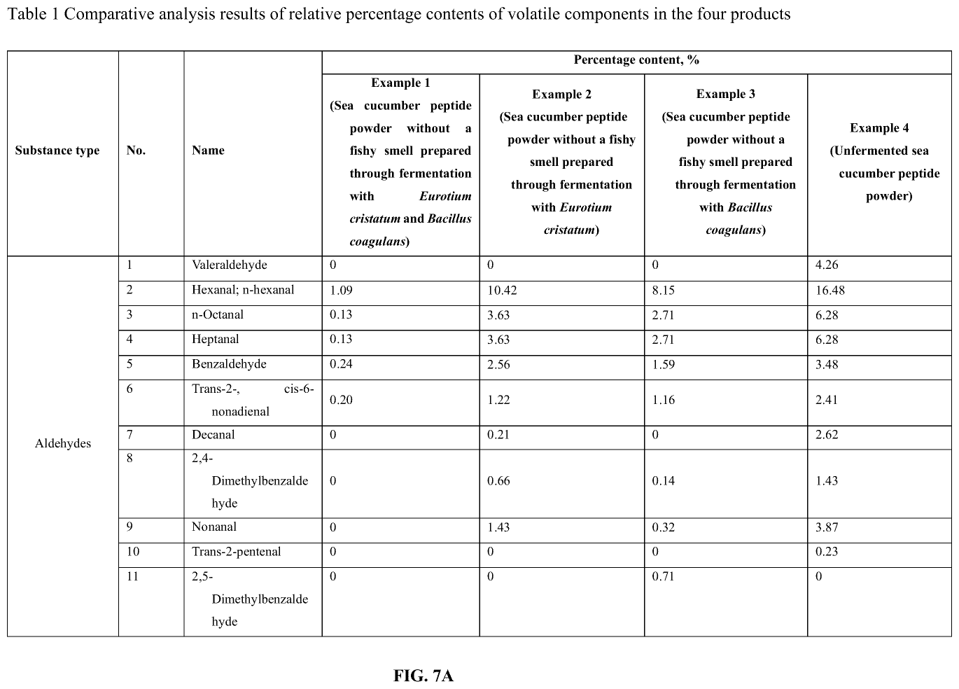

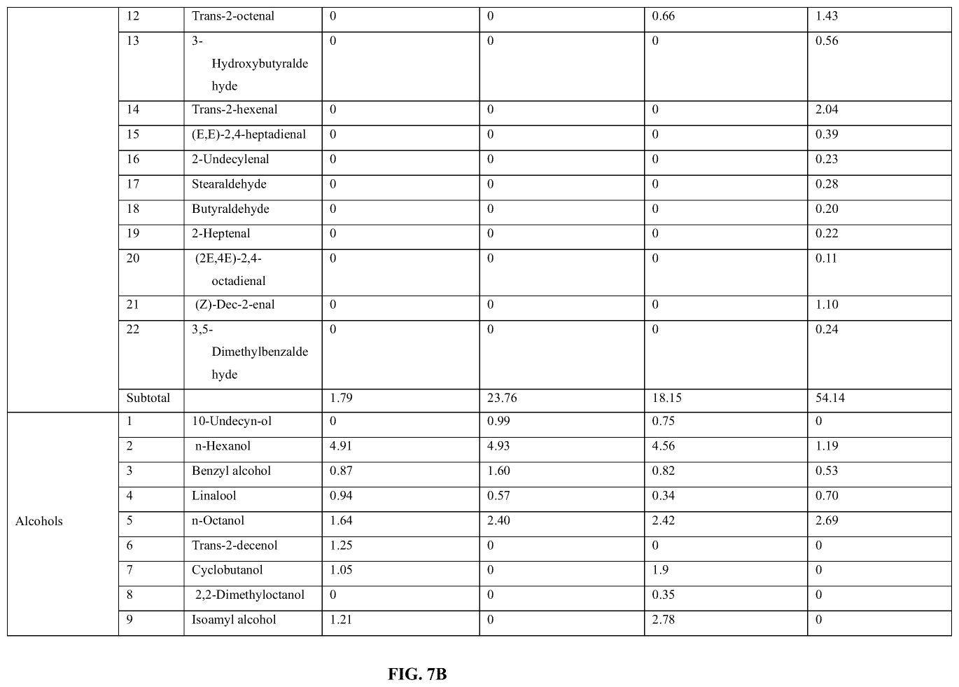

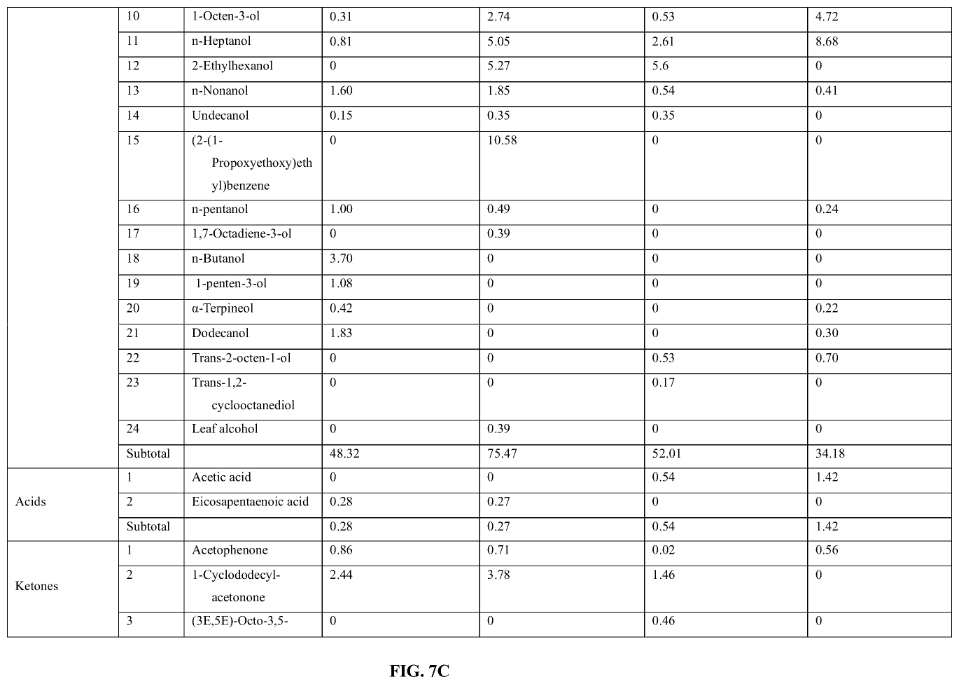

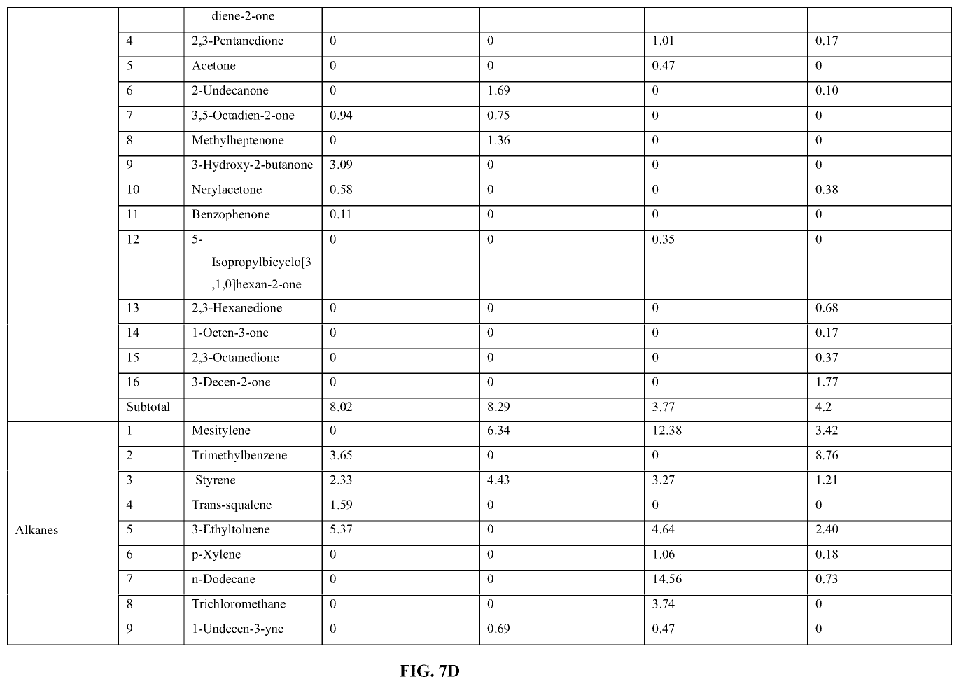

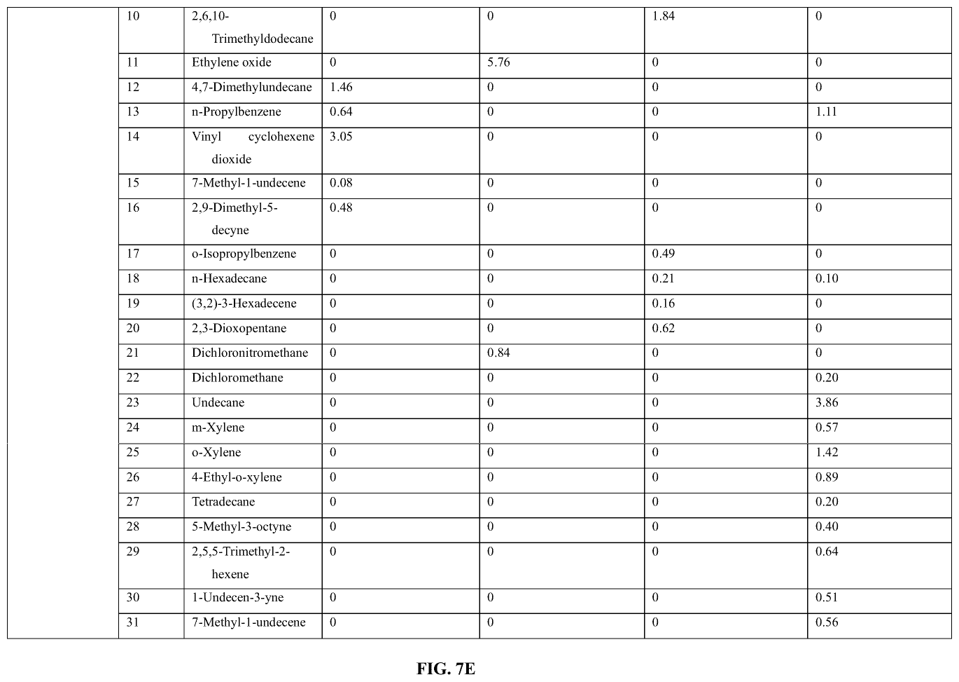

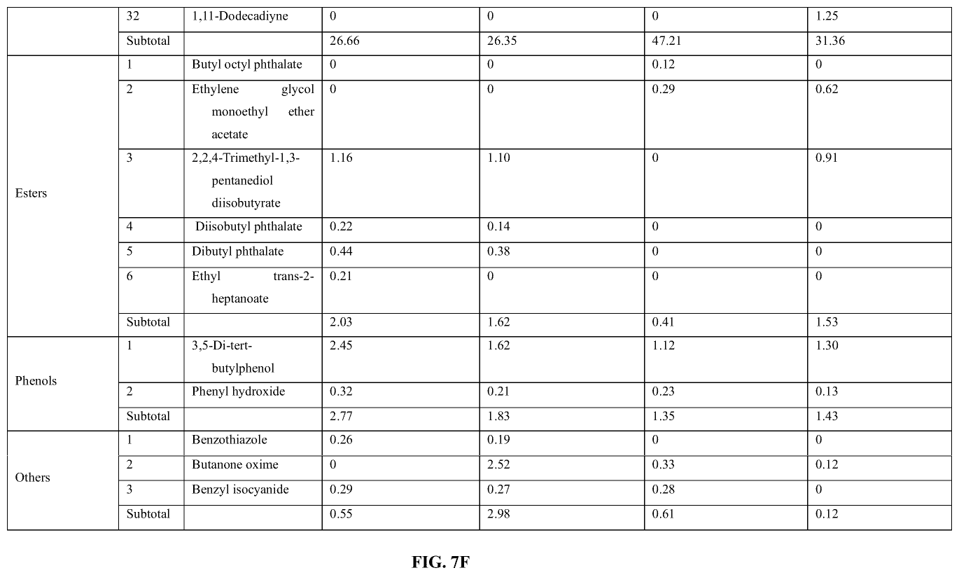

The four products in Examples 1, 2, 3, and 4 each were tested by gas chromatography-mass spectrometry (GC-MS) for volatile flavor substances. Test results are shown in Table 1 (FIG. 7), and comparative analysis results of relative percentage contents of representative fishy substances are shown in Table 2.

It can be seen from Table 1 that a total of 107 aromatic substances are detected. 65 aromatic substances are detected in the unfermented sea cucumber peptide powder in Example 4, and these substances are mainly aldehydes and alkanes. 41 aromatic substances are detected in the sea cucumber peptide powder without a fishy smell prepared through fermentation with Eurotium cristatum in Example 2, and these aromatic substances are mainly alcohols and lipids. Compared with Example 4, 5 alcohols are increased and a relative content of alcohols is increased by 2.20 times in the sea cucumber peptide powder without a fishy smell prepared through fermentation with Eurotium cristatum. 50 aromatic substances are detected in the sea cucumber peptide powder without a fishy smell prepared through fermentation with Bacillus coagulans in Example 3. 52 aromatic substances are detected in the sea cucumber peptide powder without a fishy smell prepared through fermentation with Eurotium cristatum and Bacillus coagulans in Example 1, and these aromatic substances are mainly alcohols and lipids, indicating that metabolic activities of Eurotium cristatum and Bacillus coagulans both are to produce alcohols. Major aroma characteristics of alcohols are floral, fruity, and grass aromas, and the increase of alcohols after fermentation makes sea cucumber peptides have increased pleasant aromas.

TABLE 2
Comparative analysis results of relative percentage contents of representative
fishy substances in the four sea cucumber peptide powders

| | | Percentage content, % | | | |
|---|---|---|---|---|---|
| | | | Example 2 | Example 3 | |
| | | Example 1 | (Sea cucumber | (Sea | |
| | | (Sea cucumber | peptide powder | cucumber peptide | Example 4 |
| | | peptide powder | without a fishy | powder without a | (Un- |
| | | without a fishy smell | smell prepared | fishy smell | fermented |
| | | prepared through | through | prepared through | sea |
| | | fermentation with | fermentation | fermentation with | cucumber |
| | | Eurotium cristatum and | with Eurotium | Bacillus | peptide |
| No. | Name | Bacillus coagulans) | cristatum) | coagulans) | powder) |
| 1 | Hexanal; n-hexanal | 1.09 | 8.15 | 10.42 | 16.48 |
| 2 | n-Octanal | 0.13 | 2.71 | 3.63 | 6.28 |
| 3 | Heptanal | 0.24 | 1.59 | 2.56 | 3.48 |
| 4 | Trans-2-, cis-6- | 0.20 | 1.16 | 1.22 | 2.41 |
| | nonadienal | | | | |
| 5 | Decanal | 0 | 0 | 0.21 | 2.62 |
| 6 | Trans-2-octenal | 0 | 0.14 | 0.66 | 1.43 |
| 7 | Nonanal | 0 | 0.32 | 1.43 | 3.87 |
| 8 | 1-Octen-3-ol | 0.31 | 0.53 | 2.74 | 4.72 |
| 9 | n-Heptanol | 0.81 | 2.61 | 5.05 | 8.68 |

It can be seen from Table 2 that the representative fishy substances in the unfermented sea cucumber peptide powder in Example 4 are trans-2-, cis-6-nonadienal, trans-2-octenal, decanal, nonanal, n-octanal, 1-octen-3-ol, n-hexanal, heptanal, and n-heptanol, which make the sea cucumber peptide powder have a fishy smell, an earthy taste, and a rancid smell. Compared with the unfermented sea cucumber peptide powder in Example 1, decanal is not detected in the sea cucumber peptide powder obtained through fermentation with Eurotium cristatum in Example 2. A relative content of the 9 representative fishy substances in the sea cucumber peptide powder obtained through fermentation with Eurotium cristatum in Example 2 is reduced from 49.97% to 17.21% compared with the unfermented sea cucumber peptide powder in Example 4. A relative content of the 9 representative fishy substances in the sea cucumber peptide powder obtained through fermentation with Bacillus coagulans in Example 3 is reduced from 49.97% to 27.92% compared with the unfermented sea cucumber peptide powder in Example 4. In the sea cucumber peptide powder obtained through fermentation with a mixture of Eurotium cristatum and Bacillus coagulans in Example 1, decanal and trans-2-octenal are not detected and a relative content of the 9 representative fishy substances is reduced from 49.97% to 2.78% compared with the unfermented sea cucumber peptide powder in Example 4.

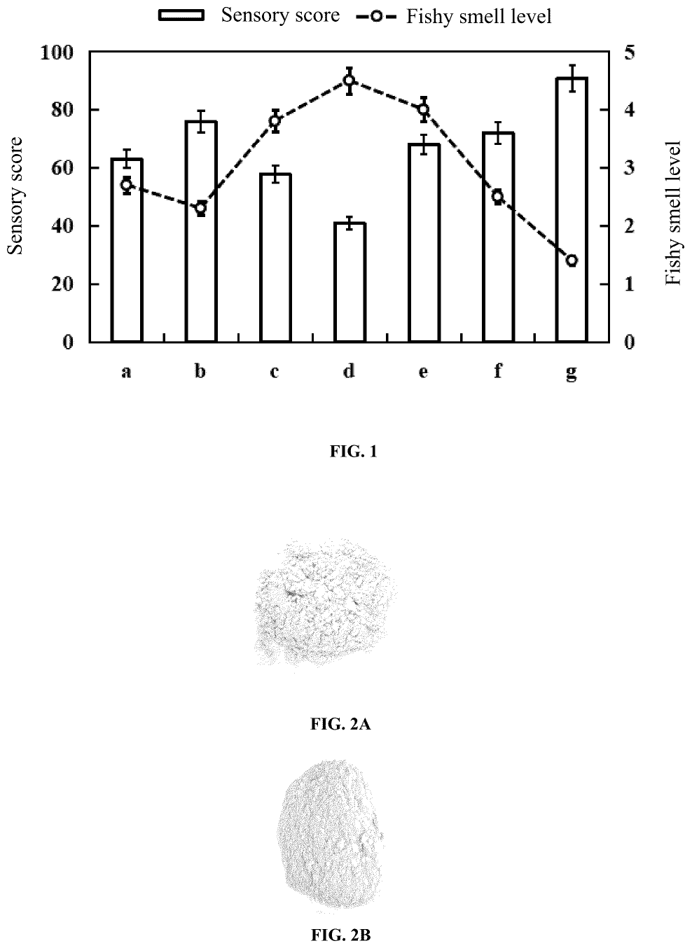

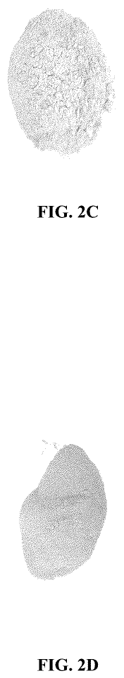

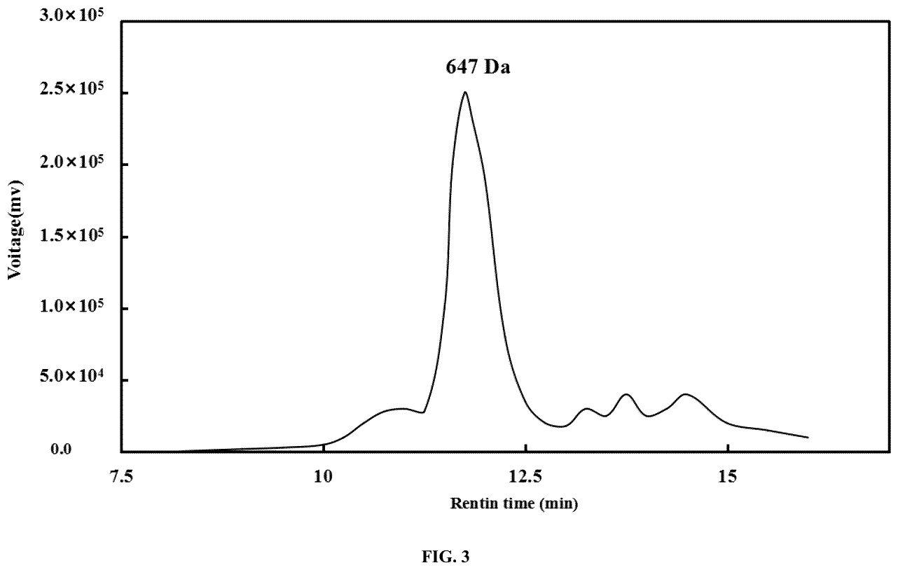

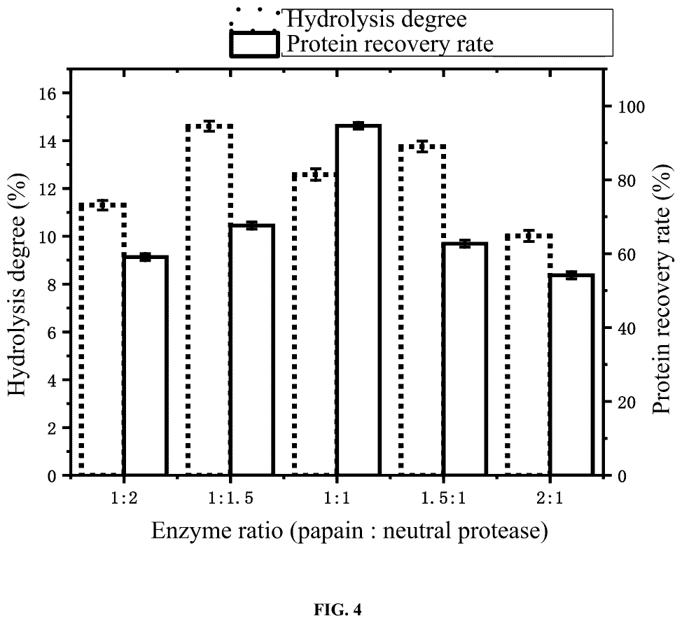

With reference to the above detection method, fishy smell levels of the sea cucumber peptide powders in Examples 1 to 7 were detected. Detection results are shown in FIG. 1.

### Example 10 Comparative Analysis of Decolorization of Sea Cucumber Peptide Powders Prepared by Different Methods

A chromaticity of each sample was determined by a WSC-S colorimeter at room temperature. L*, a*, and b* represent color coordinates, and a Hunter whiteness calculation method is adopted. L* represents a brightness of a color, and a* and b* each represent a color direction. The following standard whiteboard parameters are adopted: L*=93.61, a*=0.89, and b*=1.43. Color results of Examples 1 to 4 are shown in Table 3.

TABLE 3
Colors of the sea cucumber peptide powders in the four examples

| | Color | | |
|---|---|---|---|
| No. | L* | a* | b* |
| Example 1 | 44.62 ± 0.07d | 13.36 ± 0.12c | 30.56 ± 0.15c |
| (Sea cucumber peptide powder | | | |
| without a fishy smell prepared | | | |
| through fermentation with | | | |
| Eurotium cristatum and | | | |
| Bacillus coagulans) | | | |
| Example 2 | 40.59 ± 0.13c | 11.17 ± 0.09b | 29.88 ± 0.17bc |
| (Sea cucumber peptide powder | | | |
| without a fishy smell prepared | | | |
| through fermentation with | | | |
| Eurotium cristatum) | | | |
| Example 3 | 38.86 ± 0.12b | 11.23 ± 0.13b | 27.42 ± 0.11b |
| (Sea cucumber peptide powder | | | |
| without a fishy smell prepared | | | |
| through fermentation with | | | |
| Bacillus coagulans) | | | |
| Example 4 | 29.23 ± 0.14a | 8.75 ± 0.10a | 20.33 ± 0.09a |
| (Unfermented sea | | | |
| cucumber peptide powder) | | | |

Note:
Different letters in each column represent a significant difference between two sets of data.

It can be seen from Table 3 that the sea cucumber peptide powders obtained in different examples have different L*, a*, and b* values, indicating that a preparation method has a great impact on a color of a sea cucumber peptide powder. There is a significant difference between Example 1 and Example 4. Compared with the sea cucumber peptide powder in Example 4, the sea cucumber peptide powder without a fishy smell prepared through fermentation with Eurotium cristatum and Bacillus coagulans in Example 1 is milky-yellow with a bright luster.

### Example 11 Comparative Analysis of Impacts of Sea Cucumber Peptide Powders Prepared by Different Methods on an Immunity

An immunocompromised mouse model was established through cyclophosphamide induction, and whether the four sea cucumber peptide powder products in Examples 1, 2, 3, and 4 could improve an immunity of the immunocompromised mouse model was determined. An animal experiment was specifically as follows:

Clean-grade Kunming mice were divided into 6 groups with 12 mice in each group, including a blank group, a model group, a group of the sea cucumber peptide powder without a fishy smell prepared through fermentation with Eurotium cristatum and Bacillus coagulans in Example 1, a group of the sea cucumber peptide powder without a fishy smell prepared through fermentation with Eurotium cristatum in Example 2, a group of the sea cucumber peptide powder without a fishy smell prepared through fermentation with Bacillus coagulans in Example 3, and a group of the unfermented sea cucumber peptide powder in Example 4. An intragastric dose of each sea cucumber peptide powder was 5 mg·kg⁻¹. Mice in each experimental group were intragastrically administered once a day, and mice in the blank group and the model group were intragastrically administered with a same amount of normal saline. The administration was conducted consecutively for 14 d. On day 8, day 9, and day 10 of the animal experiment, mice in the model group and the sea cucumber peptide powder groups each were intraperitoneally injected with an 80 mg·kg⁻¹ cyclophosphamide solution consecutively for three days to establish immunocompromised mouse models. The mice were fasted and dehydrated for 12 h after the last administration, weighed, and sacrificed through dislocation, thoracic and abdominal cavities of the mice were incised, and spleens and thymuses each were collected and weighed. A spleen index and a thymus index were calculated. Calculation formulas are as follows:

$$Thymus index = \frac{T hymus weight ( mg )}{Mouse body weight ( g )}$$

$$Spleen index = \frac{S pleen weight ( mg )}{Mouse body weight ( g )}$$

The spleen and thymus are important immune organs of a human body, and changes in thymus and spleen indexes are important indexes of an immunity of a human body. Results of impacts of a sea cucumber peptide powder on body weights and organ indexes of immunocompromised mice in each example group are shown in Table 4.

TABLE 4
Impacts of the sea cucumber peptide powder in each example on body
weights and organ indexes of immunocompromised mice

| | Initial body | Final body | | |
|---|---|---|---|---|
| | weight | weight | Spleen index | Thymus index |
| Group | (g) | (g) | (mg/10 g bw) | (mg/10 g bw) |
| Blank group | 24.61 ± 0.77a | 26.71 ± 0.89b | 41.23 ± 0.92c | 28.19 ± 0.21d |
| Model group | 24.69 ± 0.67a | 18.74 ± 0.59a | 28.10 ± 0.27a | 7.97 ± 0.32a |
| Example 4 | 24.98 ± 0.64a | 19.96 ± 0.72a | 30.68 ± 0.51a | 8.46 ± 0.48a |
| (Unfermented sea | | | | |
| cucumber peptide powder) | | | | |
| Example 3 | 24.86 ± 0.75a | 20.60 ± 0.76a | 30.12 ± 0.60a | 10.14 ± 0.35b |
| (Sea cucumber peptide powder | | | | |
| without a fishy smell prepared | | | | |
| through fermentation with | | | | |
| Bacillus coagulans) | | | | |
| Example 2 | 24.21 ± 0.61a | 21.59 ± 0.90ab | 33.12 ± 0.60b | 18.14 ± 0.35c |
| (Sea cucumber peptide powder | | | | |
| without a fishy smell prepared | | | | |
| through fermentation with | | | | |
| Eurotium cristatum) | | | | |
| Example 1 | 24.79 ± 0.80a | 26.44 ± 0.90b | 40.79 ± 0.49c | 27.53 ± 0.16d |
| (Sea cucumber peptide powder | | | | |
| without a fishy smell prepared | | | | |
| through fermentation with | | | | |
| Eurotium cristatum and | | | | |
| Bacillus coagulans) | | | | |

Note:
Data are expressed as mean ± standard deviation (SD); n = 12; and different letters in each column represent a significant difference between two sets of data.

It can be seen from the results in Table 4 that there is no significant difference in an initial body weight among mice of the groups, and during the experiment, a final body weight of the model group decreases very significantly compared with a final body weight of the blank group, and final body weights of mice in the Example 1 and Example 2 groups both increase compared with the final body weight of the model group, where a final body weight of mice in the Example 1 group increases very significantly. It can be seen from the results that the sea cucumber peptide powder in Example 2 can significantly promote a weight gain of the immunocompromised mice, and the sea cucumber peptide powder in Example 1 can very significantly promote a weight gain of the immunocompromised mice.

The impacts of the sea cucumber peptide powders obtained in the examples on spleen and thymus indexes of the immunocompromised mice can be seen from Table 4. Spleen and thymus indexes of the model group both are very significantly lower than spleen and thymus indexes of the blank group, indicating that the injection of cyclophosphamide causes the atrophy of immune organs. After the immunocompromised mice are intragastrically administered with the sea cucumber peptide powders in Examples 1 to 4, spleen and thymus indexes of mice in Example 1 and Example 2 groups both increase compared with spleen and thymus indexes of mice in the model group, where a spleen index of mice in the Example 1 group increases very significantly. Therefore, it can be inferred that the sea cucumber peptide powder obtained in Example 1 can effectively alleviate the atrophy and damage of immune organs of the immunocompromised mice caused by cyclophosphamide, and has a significant immunity-improving effect.

### Example 12 Comparative Analysis of Sensory Evaluation of Sea Cucumber Peptide Powders Prepared by Different Methods

The four products in Examples 1, 2, 3, and 4 each were dissolved in water, and 12 volunteers were invited to conduct sensory evaluation. Sensory score results are shown in Table 5. The sea cucumber peptide powder prepared through fermentation with a mixture of Eurotium cristatum and Bacillus coagulans has the highest sensory score of 93.6, the sea cucumber peptide powder prepared through fermentation with Eurotium cristatum has a sensory score of 75.5, the sea cucumber peptide powder prepared through fermentation with Bacillus coagulans has a sensory score of 73.5, and the unfermented sea cucumber peptide powder has the lowest sensory score of 66.3. Specific data of the sea cucumber peptide powders without a fishy smell are shown in Table 6. With reference to the above evaluation method, the sea cucumber peptide powders in Examples 1 to 7 were subjected to sensory evaluation. Sensory evaluation results are shown in FIG. 1.

TABLE 5
Sensory scores

| Evaluation indexes | Evaluation criteria | Score |
|---|---|---|
| Color | A brown cloudy liquid | 1-5 |
| (20 points) | A light-brown transparent liquid with a | 6-9 |
| | lot of suspended solids | |
| | An orange-yellow transparent liquid | 10-15 |
| | with a small amount of suspended solids | |
| | A light-yellow transparent liquid with | 16-20 |
| | almost no suspended solids | |
| Smell | A fermented smell and a sour are too heavy | 1-5 |
| (30 points) | and are accompanied by a peculiar smell and | |
| | a fishy smell to some degree | |
| | An inherent smell of a sea cucumber retains | 6-10 |
| | and is slightly accompanied by a fishy smell | |
| | There is an inherent smell of a sea cucumber, | 11-20 |
| | a fresh fragrance produced by fermentation, | |
| | and a very weak fishy smell | |
| | There is an inherent smell of a sea cucumber, | 21-30 |
| | pleasant floral and fruity aromas, and a | |
| | refreshing sour, and there is no fishy smell. | |
| Taste | A product is difficult to swallow due to an acidity | 1-5 |
| (30 points) | and has specified rancid and fishy smells | |
| | A product has a mellow smell of fermentation, | 6-10 |
| | a heavy sour, and a slight fishy smell | |
| | A product has a boring taste, an inherent taste of | 11-20 |
| | a sea cucumber, and an inapparent mellow | |
| | smell of fermentation, and a very weak fishy smell. | |
| | A product has an inherent taste of a sea cucumber, | 21-30 |
| | a mellow smell of fermentation, and a | |
| | pure sour, and does not have a fishy smell | |
| Structure status | There are flocculents and precipitates | 1-6 |
| (20 points) | There is a small amount of flocculents | 7-14 |
| | A product is homogeneous, delicate, and free | 15-20 |
| | of flocculents and precipitates | |

TABLE 6
Sensory evaluation results of the four sea cucumber peptide powders

| | | | | Structure | Total |
|---|---|---|---|---|---|
| Product | Color | Smell | Taste | status | score |
| Example 1 | 19.2 | 28.1 | 27.6 | 18.7 | 93.6 |
| (Sea cucumber peptide powder without a | | | | | |
| fishy smell prepared through fermentation | | | | | |
| with Eurotium cristatum and Bacillus | | | | | |
| coagulans) | | | | | |
| Example 2 | 19.0 | 19.4 | 18.9 | 18.2 | 75.5 |
| (Sea cucumber peptide powder without a fishy | | | | | |
| smell prepared through fermentation with | | | | | |
| Eurotium cristatum) | | | | | |
| Example 3 | 19.1 | 18.8 | 17.3 | 18.3 | 73.5 |
| (Sea cucumber peptide powder without a | | | | | |
| fishy smell prepared through fermentation | | | | | |
| with Bacillus coagulans) | | | | | |
| Example 4 | 18.7 | 14.6 | 14.9 | 18.1 | 66.3 |
| (Unfermented sea cucumber peptide powder) | | | | | |

### Example 13 Impacts of Different Enzymatic Hydrolysis Processes to Prepare a Sea Cucumber Peptide Powder on a Protein Recovery Rate and a Hydrolysis Degree

1. Preparation of a sea cucumber peptide powder through step-by-step enzymatic hydrolysis (a neutral protease was first added, and then papain was added).

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate, which was a homogenate of the body wall of the sea cucumber for later use.

(3) Enzymatic hydrolysis: The filtrate obtained in the step (2) was added to a beaker and subjected to step-by-step enzymatic hydrolysis with an enzyme combination as follows: A neutral protease was first added to allow first enzymatic hydrolysis for 1 h at 50° C. and a pH of 7; and then papain was added to allow second enzymatic hydrolysis for 1.5 h at 50° C. and a pH of 7, and a resulting supernatant was collected, which was an enzymatic hydrolysate. A total amount of the neutral protease and the papain was 8,000 U/g, and an enzymatic activity ratio of the papain to the neutral protease was 1:1.

2. Preparation of a sea cucumber peptide powder without a fishy smell through step-by-step enzymatic hydrolysis (papain was first added, and then a neutral protease was added).

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate.

(3) Enzymatic hydrolysis: The filtrate obtained in the step (2) was added to a beaker and subjected to step-by-step enzymatic hydrolysis with an enzyme combination as follows: Papain was first added to allow first enzymatic hydrolysis for 1.5 h at 50° C. and a pH of 7; and then a neutral protease was added to allow second enzymatic hydrolysis for 1 h at 50° C. and a pH of 7, and a resulting supernatant was collected, which was an enzymatic hydrolysate. A total amount of the neutral protease and the papain was 8,000 U/g, and an enzymatic activity ratio of the papain to the neutral protease was 1:1.

3. Preparation of a sea cucumber peptide powder without a fishy smell through synergistic enzymatic hydrolysis (a neutral protease and papain were added simultaneously).

(1) Pretreatment: A dried sea cucumber was washed and soaked in water at room temperature for 24 h during which the water was changed every 4 h, and then inner and outer surfaces of a body wall of a washed sea cucumber were cleaned with clean water.

(2) Beating: The body wall of the sea cucumber obtained after the step (1) was drained and cut into small pieces of (1-2) cm×(1-2) cm, water was added, and beating was conducted to obtain a slurry; and the slurry was homogenized and filtered to obtain a filtrate.

(3) Enzymatic hydrolysis: The filtrate obtained in the step (2) was added to a beaker, the papain and the neutral protease were added simultaneously to allow synergistic enzymatic hydrolysis for 3 h at 50° C. and a pH of 7, and a resulting supernatant was collected, which was an enzymatic hydrolysate. A total amount of the neutral protease and the papain was 8,000 U/g, and an enzymatic activity ratio of the papain to the neutral protease was 1:1.

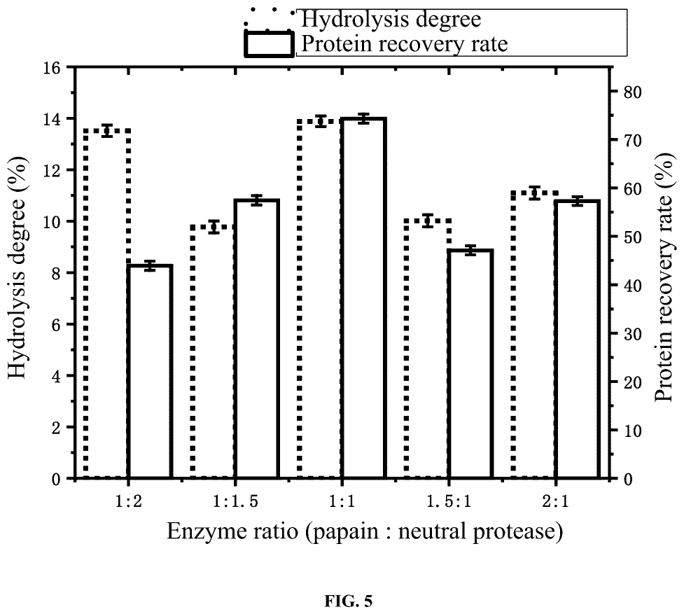

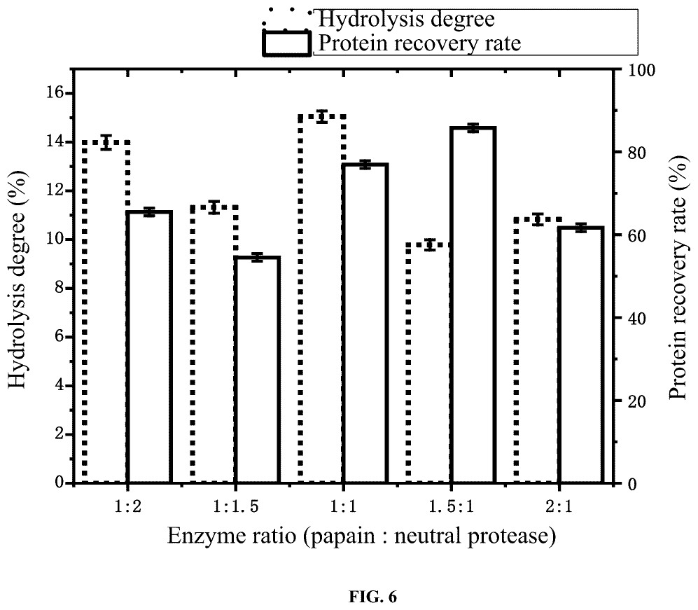

For the above three enzymatic hydrolysates, a hydrolysis degree was determined by an o-phthalaldehyde (OPA) method, and a protein recovery rate was determined by a Kjeldahl nitrogen determination method. Specific data are detailed in FIG. 4 to FIG. 6. It can be seen from FIG. 4 that, when the neutral protease is added first and then the papain is added with a ratio of the papain to the neutral protease being 1:1.5 or 1:1, a recovery rate and a hydrolysis degree of sea cucumber proteins are 14.8% and 79% or 12.9% and 97%, respectively. It can be seen from FIG. 5 that, when the papain is first added and then the neutral protease is added with a ratio of the papain to the neutral protease being 1:1, a hydrolysis degree and a recovery rate of sea cucumber proteins are 14.3% and 76%, respectively. FIG. 6 shows that, when the neutral protease and the papain are added simultaneously with a ratio of the papain to the neutral protease being 1:1, a hydrolysis degree and a recovery rate of sea cucumber proteins are 14.6% and 81%, respectively. According to comprehensive consideration of the protein recovery rates and hydrolysis degrees, the optimal conditions are as follows: the neutral protease is added first and then the papain is added, and the ratio of the papain to the neutral protease is 1:1.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A preparation method of a probiotic-containing sea cucumber peptide powder for improving an immunity, comprising the following steps:
(1) to a homogenate of a body wall of a sea cucumber, adding a neutral protease to allow first enzymatic hydrolysis, and then adding papain to allow second enzymatic hydrolysis to obtain an enzymatic hydrolysate; and
(2) adding a probiotic to the enzymatic hydrolysate to allow fermentation, and collecting a supernatant produced after the fermentation,
wherein the probiotic in the step (2) is Eurotium cristatum with an accession number of CICC No. 2099 and/or Bacillus coagulans with an accession number of CGMCC No. 21801.

2. The preparation method of a probiotic-containing sea cucumber peptide powder for improving an immunity according to claim 1, wherein in the step (1), an enzymatic activity ratio of the neutral protease to the papain is (1-5):1.

3. The preparation method of a probiotic-containing sea cucumber peptide powder for improving an immunity according to claim 1, wherein in the step (1), a total amount of the neutral protease and the papain is 6,000 U/g to 9,000 U/g, and the first and second enzymatic hydrolysis are conducted for 3 h to 5 h in total at 50° C. to 55° C. and a pH of 6.5 to 7.5.

4. The preparation method of a probiotic-containing sea cucumber peptide powder for improving an immunity according to claim 1, wherein in the step (2), the fermentation is conducted at 36° C. to 38° C. for 40 min to 60 min.

5. The preparation method of a probiotic-containing sea cucumber peptide powder for improving an immunity according to claim 1, wherein in the step (2), a colony-forming unit (CFU) ratio of the Eurotium cristatum to the Bacillus coagulans is 1:(1-3), and a total amount of the Eurotium cristatum and the Bacillus coagulans is 0.02% to 0.04% of a mass of the enzymatic hydrolysate.

6. The preparation method of a probiotic-containing sea cucumber peptide powder for improving an immunity according to claim 1, wherein in the step (2), an amount of the Eurotium cristatum is 0.02% to 0.04% of a mass of the enzymatic hydrolysate.

7. The preparation method of a probiotic-containing sea cucumber peptide powder for improving an immunity according to claim 1, wherein in the step (2), an amount of the Bacillus coagulans is 0.02% to 0.04% of a mass of the enzymatic hydrolysate.

8. The preparation method of a probiotic-containing sea cucumber peptide powder for improving an immunity according to claim 1, wherein in the step (2), the Eurotium cristatum and the Bacillus coagulans are added as follows: the Eurotium cristatum and the Bacillus coagulans are first mixed and then added to the enzymatic hydrolysate in the step (1) to allow the fermentation; or the Eurotium cristatum and the Bacillus coagulans are added separately to the enzymatic hydrolysate in the step (1) to allow the fermentation.

9. The preparation method of a probiotic-containing sea cucumber peptide powder for improving an immunity according to claim 1, further comprising: concentrating the supernatant collected in the step (2), and spray-drying a concentrate at a low temperature to obtain the probiotic-containing sea cucumber peptide powder.

10. A probiotic-containing sea cucumber peptide powder for improving an immunity prepared by the preparation method according to claim 1.

11. The probiotic-containing sea cucumber peptide powder for improving an immunity according to claim 10, wherein in the step (1), an enzymatic activity ratio of the neutral protease to the papain is (1-5):1.

12. The probiotic-containing sea cucumber peptide powder for improving an immunity according to claim 10, wherein in the step (1), a total amount of the neutral protease and the papain is 6,000 U/g to 9,000 U/g, and the first and second enzymatic hydrolysis are conducted for 3 h to 5 h in total at 50° C. to 55° C. and a pH of 6.5 to 7.5.

13. The probiotic-containing sea cucumber peptide powder for improving an immunity according to claim 10, wherein in the step (2), the fermentation is conducted at 36° C. to 38° C. for 40 min to 60 min.

14. The probiotic-containing sea cucumber peptide powder for improving an immunity according to claim 10, wherein in the step (2), a colony-forming unit (CFU) ratio of the Eurotium cristatum to the Bacillus coagulans is 1:(1-3), and a total amount of the Eurotium cristatum and the Bacillus coagulans is 0.02% to 0.04% of a mass of the enzymatic hydrolysate.

15. The probiotic-containing sea cucumber peptide powder for improving an immunity according to claim 10, wherein in the step (2), an amount of the Eurotium cristatum is 0.02% to 0.04% of a mass of the enzymatic hydrolysate.

16. The probiotic-containing sea cucumber peptide powder for improving an immunity according to claim 10, wherein in the step (2), an amount of the Bacillus coagulans is 0.02% to 0.04% of a mass of the enzymatic hydrolysate.

17. The probiotic-containing sea cucumber peptide powder for improving an immunity according to claim 10, wherein in the step (2), the Eurotium cristatum and the Bacillus coagulans are added as follows: the Eurotium cristatum and the Bacillus coagulans are first mixed and then added to the enzymatic hydrolysate in the step (1) to allow the fermentation; or the Eurotium cristatum and the Bacillus coagulans are added separately to the enzymatic hydrolysate in the step (1) to allow the fermentation.
